(19) United States
(12) Patent Application Publication
(10) Pub. No.: US 2003/0119028 A1
(43) Pub. Date: Jun. 26, 2003

(54) Device and methods for enhanced microarray hybridization reactions

(76) Inventors: David J. Graves, Devon, PA (US); Paolo Fortina, Philadelphia, PA (US); Richard (Hung-Ju) Su, Arcadia, CA (US)

Correspondence Address: DANN DORFMAN HERRELL & SKILLMAN, SUITE 720, 1601 MARKET STREET, PHILADELPHIA, PA, 19103-2307, US

(21) Appl. No.: 10/213,770

(22) Filed: Aug. 7, 2002

Related U.S. Application Data
(60) Provisional application No. 60/310,766, filed on Aug. 8, 2001.

(51) Int. Cl. C12Q 1/68; C12M 1/34

(52) U.S. Cl. 435/6; 435/287.2

(57) ABSTRACT

A device and methods are provided for enhancing DNA microarray hybridization speed and discrimination efficiency by means of an electric field.

### CROSS-REFERENCE TO RELATED APPLICATION

This application claims the benefit of U.S. Provisional Application No. 60/310,766, filed Aug. 8, 2001, the entire disclosure of which is hereby incorporated by reference.

### FIELD OF THE INVENTION

This invention relates generally to DNA microarray technology, and more specifically to devices and methods that enhance the speed and discrimination of nucleic acid hybridization reactions.

### BACKGROUND OF THE INVENTION

DNA microarray technology has emerged as a powerful tool for discovering genetic information. The application of this revolutionary technology, embodied in what are known as DNA chips, has resulted in explosive discoveries in the fields of health-related sciences and medicine. The major applications of DNA microarrays are divided in the two categories: studies of genomic structure and studies of active gene expression. The former includes genetic disease diagnosis (e.g., mutation detection), polymorphism analysis (e.g., SNP analysis), gene mapping, and sequencing by hybridization. The latter mainly provides information about which genes are currently active in a given sample and at what level. Such information aids in understanding the phenotype of an organism, which determines its form and function.

In its most basic form, a DNA microarray is simply a solid support, e.g. glass or silicon, bearing on its surface an array of different DNA fragments (called “probes”), usually having a known sequence, at discrete locations or spots on the support. The DNA spots on the chip are hybridized to detectably labeled nucleic acid molecules (called “targets”) which are present in a test sample. The pattern and extent of detectable label, e.g. fluorescence, that is observed provides information about the nucleic acids present in the solution, either qualitatively in searching for the presence of a particular sequence (for example, mutation detection), or quantitatively, in attempting to determine the amount of numerous sequences likely to be present (as in gene expression patterns).

Microhybridization arrays on glass slides enable heterogeneous hybridization between the target nucleic acids and the probes. Each microarray consists of several hundred to several hundred thousand microscopic spots. Each spot in the array contains identical, single strand oligonucleotide probes which are usually 10-30 bases long or complementary DNA (cDNA) probes, typically 500-1,000 bases long. The amount of the probe attached to the solid support is small and the spots are closely spaced. Thus, the consumption of probe solution to make spots and the volume of target-containing test solution are both low. The probes are attached to the solid support by chemical linkage or chemisorption. A solution phase of oligonucleotides or single stranded DNA labelled with a detectable reporter is then poured onto the support surface. Only two complementary strands, one in the liquid phase and the other on the solid phase, will hybridize under appropriate conditions of hybridization and washing. The support is then brought to a suitable detection instrument to determine the degree of hybridization.

DNA microarray technology has many advantages in comparison to previous methods such as Southern blotting. First, microarrays enable performing analyses in parallel. Arrays consist of a large variety of different DNA spots, and a corresponding number of targets can be tested for simultaneously. Second, microarrays use very little material. Since microarrays are compact, only a small amount of biological sample is consumed, thereby reducing the cost substantially. Third, microarrays require only a limited investment for labor. Most parts of the process for generating DNA microarrays are automated and high-throughput in nature, reducing human involvement.

One of the main differences between DNA microarrays and Southern blotting that influences the hybridization process is in the use of an impermeable, solid substrate, usually glass, instead of the membrane support used in Southern blotting. Additionally, the positions of the probes and targets are reversed, i.e., in Southern blotting, the targets are disposed on the support, and the probes are in solution. The solid glass support has a number of advantages over porous membranes used in Southern blotting. The main advantage is that target molecules cannot penetrate the surface. Therefore, target nucleic acid molecules have immediate access to the probes once they contact the glass surface. In addition, the washing step following the spotting or hybridization step for removing unbound probes or unhybridized targets is also unimpeded, thereby improving hybridization reproducibility.

Although microarray technology has many advantages compared to other existing methods, as noted above, one of the inconveniences of microarray hybridization is that the investigator typically must wait 10 to 20 hours or even 1 to 2 days for the proper hybrids to form. Because the microscopic volume of the hybridization solution on the support cannot be stirred to facilitate the reaction, mass transfer is diffusive. This is especially true for relatively long target DNA molecules because the diffusion of the longer target molecules in the solution is much slower than that of shorter ones.

Another problem is encountered in attempting to achieve hybridization between probe oligonucleotides and short target oligonucleotides. Since both perfectly matched and single-base mismatched oligonucleotides will hybridize to the probes, the matched and mismatched hybrids are formed nearly at the same rate, unless the temperature is controlled between the melting temperatures of the two hybrids. Consequently, discrimination between the two hybrids is very sensitive and usually not easily accomplished.

### SUMMARY OF THE INVENTION

In accordance with the present invention, a device for enhancing the speed and discrimination of nucleic acid hybridization reactions is provided. The device includes a solid support and a continuous barrier disposed on and surrounding a predetermined surface area on the solid support. The barrier and the predetermined surface area of the solid support surrounded by the barrier define a reaction space within the barrier. A portion of the support surface within the reaction space bears a first electrode, comprising a coating of electrically conductive material, and a micro-array of nucleic acid probes. A removable cover having a surface which cooperates with the barrier serves to enclose the reaction chamber. A portion of the surface of the cover within the reaction space bears a second electrode, comprising a coating of electrically conductive material. The device operates using a source of electric potential, including a positive pole and a negative pole, with the positive pole connected to the first electrode and the negative pole connected to the second electrode.

In a preferred embodiment of the invention, the solid support and the cover are Indium/Tin-Oxide-coated transparent slides.

In accordance with another aspect of the invention, a method is provided for enhancing the speed of nucleic acid hybridization reactions using the above-described nucleic acid hybridization device. This method comprises depositing into the reaction space of the device a volume of test sample suspected of containing target nucleic acid molecules complementary to the nucleic acid probes spotted on the support surface, enclosing the reaction space with a cover, applying an electrical potential across the electrodes of the device, the first electrode being positive and the second electrode being negative, and detecting the occurrence of hybridization reaction between the spotted nucleic acid probes and the target nucleic acid molecules.

In yet another aspect of the invention, a method for improving discrimination efficiency between hybrids formed by the reaction between nucleic acid probes and perfectly matched target nucleic acid molecules and nucleic acid probes and target nucleic acid that differ from the nucleic acid probes by at least one mismatched base pair is provided. This method comprises (a) providing a hybridization device, as described above, (b) depositing into the reaction space of the device a volume of test sample containing target nucleic acid molecules comprising perfectly matched nucleic acids and nucleic acids having at least one mismatched base pair, (c) subjecting the contents of the reaction space to conditions promoting hybridization between the nucleic acid probes and the target nucleic acid molecules, (d) enclosing the reaction space with the cover, (e) applying a potential difference to the electrodes, so that the first electrode is positive and the second electrode is negative, and for a time sufficient to effect dissociation of a fraction of the hybrids formed in step c, (f) reversing the potential difference applied to the first and second electrodes in step e, (g) restoring the potential difference applied to the first and second electrodes in step e, and (h) determining the level of hybrids formed between the nucleic acid probes and the perfectly matched nucleic acids, in relation to hybrids formed between the nucleic acid probes and the nucleic acids having at least one mismatched base pair and comparing the level to the corresponding level of hybridization in step c.

### BRIEF DESCRIPTION OF THE DRAWINGS

FIG. 1 shows the 865 base sense strand PCR target sequence of human Connexin 26 (SEQ ID NO: 1). Italic G represents the 35 deletion and T represents the 167 deletion. The underlined sequence represents the forward PCR Primer.

FIG. 2 shows the 865 base antisense strand PCR target sequence of human Connexin 26 (SEQ ID NO: 2). Underlined sequences represent the reverse PCR primers.

FIG. 3 is a diagram illustrating the relative position of all four PCR sense strand targets within the Connexin 26 gene as well as the relative location where the probes hybridize.

FIG. 4 is a diagram illustrating an electric field hybridization device according to the invention, which enables improved hybridization speed. A double-sided adhesive chamber is attached to the indium/Tin-Oxide (ITO)-coated glass slide that acts as the positive electrode. Nucleic acid probes are spotted on the surface of the glass slide. Hybridization solution containing target molecules is pipetted into the chamber, and the chamber is sealed with a second ITO-coated slide that acts as the negative electrode. The (+) and (−) indicate the charge of the electrodes when attached to a voltage source (not shown).

FIG. 5 is a diagram illustrating the DNA spotting pattern for the hybridization reactions. Each probe is spotted in a row of five duplicate spots, except for Row 7 which has seven spots for orientation purposes. Black spots (Rows 1 and 7) represent the positive controls and gray spots (Row 4) represent the negative control. Rows 2, 3, 5 and 6 are probes for capturing various sizes of complementary PCR targets.

FIG. 6 shows the microarray image of the results of the electric-field hybridization reaction using 100 fmole of denatured 157 base PCR product (sense and antisense) and equal quantities of hybridization control targets. Rows 1 and 7 are the positive controls, and Row 4 is the negative control. Hybridization of the sense strand PCR target was detected in Row 3.

FIG. 7 shows the microarray image of the results of the electric-field hybridization reaction using 100 fmole of denatured 323 base PCR product (sense and antisense) and equal quantities of hybridization control targets. Rows 1 and 7 are the positive controls, and Row 4 is the negative control. Hybridization of the antisense PCR targets were detected in Rows 2 and 5.

FIG. 8 shows the microarray image of the results of the electric-field hybridization reaction using 100 fmole of denatured 651 base PCR product (sense and antisense) and equal quantities of hybridization control targets. Rows 1 and 7 are the positive controls, and Row 4 is the negative control. Hybridization of the sense strand PCR target was detected in Rows 3 and 6.

FIG. 9 shows the microarray image of the results of the electric-field hybridization reaction using 100 fmole of denatured 864 base PCR product (sense and antisense) and equal quantities of hybridization control targets. Rows 1 and 7 are the positive controls, and Row 4 is the negative control. Hybridization of the sense strand PCR target was detected in Rows 3 and 6.

FIG. 10 shows the microarray image of the results of a passive hybridization reaction using 100 fmole of denatured 157 base PCR product (sense and antisense) and equal quantities of hybridization control targets. Rows 1 and 7 are the positive controls, and Row 4 is the negative control. Hybridization of the sense strand PCR target was detected in Row 3.

FIG. 11 is a diagram illustrating the reversed electric-field hybridization apparatus used to enhance discrimination efficiency. The ITO-coated probe support is the negative electrode, whereas the second ITO-coated slide acts as the positive electrode. The (+) and (−) indicate the charge of the electrodes when attached to a voltage source (not shown).

FIG. 12 shows the microarray image of the results of the reversed electric-field hybridization reaction performed to enhance discrimination efficiency. Row 2 is the negative control, and Row 4 is the attachment control. Row 1 shows signal from the perfectly matched hybrids, and Row 3 shows signal from the single-base mismatched hybrids.

### DETAILED DESCRIPTION OF THE INVENTION

Although DNA microarrays are becoming important tools for carrying out molecular biological reactions, the time needed to complete the analysis (10 to 20 hours) can be significant. Thus, in accordance with the present invention, a device and methods of use have been developed which accelerate DNA microarray hybridization reactions. The hybridization reactions are accelerated by applying an electric-field to the surface of glass slides which enhances hybridization between the immobilized probe and solution phase target molecules.

In a preferred embodiment of the invention, a microarray device is provided comprised of two glass microscope slides coated with a transparent layer of indium/tin oxide (ITO) and a thin chamber or reaction space situated in between the two slides. The entire surface of the microscope slides function as single electrodes, and arrays are deposited on the surface of one slide to engage in hybridization reactions.

In another embodiment of the invention, nucleic acid hybridization reactions are performed using the following steps: (1) attaching nucleic acid spots to the conductive surface of one slide which functions as a first electrode, (2) placing a small volume of solution (approximately 25 μl) containing the complementary target molecules on the surface within a defined reaction space, (3) enclosing the reaction space with a second glass slide having a conductive surface facing the reaction space, which functions as a second electrode, (4) applying voltage across the electrodes such that the first electrode array is positive with respect to the second electrode, and (5) thereafter, disassembling the hybridization apparatus and quantitating the hybridization reactions.

This novel hybridization technique is advantageous over existing methods for several reasons: (1) the glass supports act as single electrodes which eliminates the unnecessarily complex step of having to place individual DNA spots on individual electrodes, (2) hybridization reactions may be carried out using microliter volumes of solution, (3) low concentrations of nucleic acid molecules may be detected, and (4) nucleic acid hybridization reactions occur in dramatically shorter periods of time.

In yet another embodiment of the invention, the microarray apparatus may be used to advantage to discriminate between perfectly matched and single-base mismatched hybrids. After applying the electric current to the glass slide, the current is reversed for a short time (a few seconds) to remove weaker bound single-base mismatched hybrids.

Definitions:

The following definitions are provided to facilitate an understanding of the present invention:

With reference to nucleic acids used in the invention, the term “isolated nucleic acid” is sometimes employed. This term, when applied to DNA, refers to a DNA molecule that is separated from sequences with which it is immediately contiguous (in the 5′ and 3′ directions) in the naturally occurring genome of the organism from which it was derived. An “isolated nucleic acid molecule” may also comprise a cDNA molecule or a recombinant nucleic acid molecule.

When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it would be associated in its natural state (i.e., in cells or tissues). An isolated nucleic acid (either DNA or RNA) may further represent a molecule produced directly by biological or synthetic means and separated from other components present during its production.

The term “oligonucleotide,” as used herein refers to sequences and probes of the present invention, and is defined as a nucleic acid molecule comprised of two or more ribo- or deoxyribonucleotides, preferably more than three. The exact size of the oligonucleotide will depend on various factors and on the particular application and use of the oligonucleotide.

With respect to single stranded nucleic acids, particularly oligonucleotides, the term “specifically hybridizing” refers to the association between two single-stranded nucleotide molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA molecule of the invention, to the substantial exclusion of hybridization of the oligonucleotide with single-stranded nucleic acids of non-complementary sequence. Appropriate conditions enabling specific hybridization of single stranded nucleic acid molecules of varying complementarity are well known in the art.

For instance, one common formula for calculating the stringency conditions required to achieve hybridization between nucleic acid molecules of a specified sequence homology is set forth below (Sambrook et al., 1989):

Tₘ=81.5° C.+16.6 Log[Na+]+0.41(% G+C)−0.63(% formamide)−600/#bp in duplex

As an illustration of the above formula, using [Na+]=[0.368] and 50% formamide, with GC content of 42% and an average probe size of 200 bases, the Tₘ is 57° C. The Tₘ of a DNA duplex decreases by 1-1.5° C. with every 1% decrease in homology. Thus, targets with greater than about 75% sequence identity would be observed using a hybridization temperature of 42° C.

The term “probe” as used herein refers to an oligonucleotide, polynucleotide or DNA molecule, whether occurring naturally as in a purified restriction enzyme digest or produced synthetically, which is capable of annealing with or specifically hybridizing to a nucleic acid with sequences complementary to the probe. The probes of the present invention refer specifically to the oligonucleotides attached to a solid support in the DNA microarray apparatus such as the glass slide. A probe may be either single-stranded or double-stranded. The exact length of the probe will depend upon many factors, including temperature, source of probe and use of the method. For example, for diagnostic applications, depending on the complexity of the target sequence, the oligonucleotide probe typically contains 15-25 or more nucleotides, although it may contain fewer nucleotides. The probes herein are selected to be complementary to different strands of a particular target nucleic acid sequence. This means that the probes must be sufficiently complementary so as to be able to “specifically hybridize” or anneal with their respective target strands under a set of pre-determined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically.

The term “specific binding pair” as used herein includes antigen-antibody, receptor-hormone, receptor-ligand, agonist-antagonist, lectin-carbohydrate, nucleic acid (RNA or DNA) hybridizing sequences, Fc receptor or mouse IgG-protein A, avidin-biotin, streptavidin-biotin, amine-reactive agent-amine conjugated molecule and thiol-gold interactions. Various other determinant-specific binding substance combinations are contemplated for use in practicing the methods of this invention, such as will be apparent to those skilled in the art.

The term “detectably label” is used herein to refer to any substance whose detection or measurement, either directly or indirectly, by physical or chemical means, is indicative of the presence of the target bioentity in the test sample. Representative examples of useful detectable labels, include, but are not limited to the following: molecules or ions directly or indirectly detectable based on light absorbance, fluorescence, reflectance, light scatter, phosphorescence, or luminescence properties; molecules or ions detectable by their radioactive properties; molecules or ions detectable by their nuclear magnetic resonance or paramagnetic properties. Included among the group of molecules indirectly detectable based on light absorbance or fluorescence, for example, are various enzymes which cause appropriate substrates to convert, e.g., from non-light absorbing to light absorbing molecules, or from non-fluorescent to fluorescent molecules.

Polymerase chain reaction (PCR) has been described in U.S. Pat. Nos. 4,683,195, 4,800,195, and 4,965,188, the entire disclosures of which are incorporated by reference herein.

The following examples are provided to illustrate certain embodiments of the invention. They are not intended to limit the invention in any way.

### EXAMPLE 1

### Enhanced Nucleic Acid Microarray Hybridization Reactions

DNA microarray hybridization reactions were accelerated using an electric-field as described in detail below.

I. Materials and Methods:

A. Preparation of Probes and Targets

Short probes and targets were commercially synthesized and purified by HPLC (Integrated DNA Technologies, Coralville, Iowa). Long target DNA molecules were generated by PCR from genomic DNA.

Oligonucleotides complementary to the FCyIIA gene were generated for use as positive controls. The first synthesized probe, FC3, was 21 bases long, amino-modified at the 3′ end and fluorescently labeled with Cy5 at the 5′ end. This probe was used as the attachment control. Another probe, FC2, was synthesized as a hybridization control probe. FC2 was 21 bases long and amino-modified at the 5′ end. A complementary target, FC3A, was also generated and labeled with Cy5 for hybridizing with the FC2 probe. Another probe, FC2SBPM, which was 21 bases long with 5′ end amino modification was also synthesized. This probe differed by one base from FC2. A thymine in the middle of the sequence was changed to a cysteine. FC2 and FC2SBPM were used in combination with FC3A to test the ability to discriminate between perfectly matched and single-base mismatched hybrids.

A negative control probe, mOCT1-01, was also synthesized. This probe was 20 bases long and amino modified at the 5′ end and was complementary to the mouse OCT-1 gene.

A set of oligonucleotide probes complementary to the human Connexin 26 gene was also synthesized. Four probes were generated, two at each mutation site, complementary to the Connexin 26 gene. The first two probes, 35DELGS and 35DELGA, corresponded to the mutation site, 35 deletion G where the guanine base was deleted. Both probes were 23 bases long with amino modification at the 5′ end and their sequences stopped one base short of the mutation site. These probes were used for capturing perfectly complementary PCR targets of various sizes generated from genomic DNA. 35DELGS was complementary to the antisense strand of the PCR product, while 35DELGA was complementary to sense strand. In addition, two other probes, 167DELTS and 167DELTA, were synthesized. These probes were complementary to the mutation site, 167 deletion T, where the thymine base was deleted. 167DELTS was complementary to the antisense strand and 167DELTA was complementary to the sense strand. These four probes were used to investigate the effects of various sized targets and how they influence the speed of hybridization.

Tables I and II encompass all of the probe and target molecules described herein.

TABLE I

| | | Function and |
|---|---|---|
| Name | Sequence, modification, label | description |
| FC3 | 5′ - Cy5 - TTT GGA TCC CAC CTT CTC CAT - | Probe, attachment |
| | Spacer molecules-Amino modification - | control |
| | 3′ | |
| FC2 | 5′ - Amino modification - Spacer | Probe, perfectly |
| | molecules- ATG GAG AAG GTG GGA TCC AAA - | matched to FC3A |
| | 3′ | |
| FC3A | 5′ - Cy5 - TTT GGA TCC CAC CTT CTC CAT - | Target, hybridization |
| | 3′ | control |
| FC2SBPM | 5′ - Amino modification - Spacer | Probe, single base pair |
| | molecules- ATG GAG AAG GCG GGA TCC AAA - | mismatched (SBPM) to |
| | 3′ | FC3A |
| mOCT1-01 | 5′ - Amino modification- Spacer | Probe, negative control |
| | molecules - CAA CTC GCA CAA TAG CAG CA - | |
| | 3′ | |
| 35DELGS | 5′ - Amino modification - Spacer | Probe, perfectly |
| | molecules- ACG CTG CAG ACG ATC CCT GGG | matched to Connexin 26 |
| | GG- 3′ | PCR antisense strand |
| 35DELGA | 5′ - Amino modification - Spacer | Probe, perfectly |
| | molecules- GTG GAG TGT TTG TTC ACA CCC | matched to Connexin 26 |
| | CC - 3′ | PCR sense strand |
| 167DELTS | 5′ - Amino modification - Spacer | Probe, perfectly |
| | molecules-AGG CCG ACT TTG TCT GCA ACA | matched to Connexin 26 |
| | CCC- 3′ | PCR antisense strand |
| 167DELTA | 5′ - Amino modification - Spacer | Probe, perfectly |
| | molecules- CAC ACG TTC TTG CAG CCT GGC | matched to Connexin 26 |
| | TGC - 3′ | PCR sense strand |
| Connexin 26 | 157, 323, 651 and 864 bp PCR product | Target, biotinylated |
| PCR | | |

Four longer DNA target molecules, both sense and antisense strands, were prepared by PCR from the human Connexin 26 gene (See Table II). These target molecules were used to study the effect of target length on the speed of hybridization.

TABLE II

| PCR | Forward primer sequence | Reverse primer sequence |
|---|---|---|
| product | (Name) | (Name) |
| 157 bases | 5′ gca ttc gtc ttt tcc aga gc | 5′ cag cca caa cga gga tca ta |
| | (CX26CD1S) | (CX26-316R) |
| 323 bases | 5′ gca ttc gtc ttt tcc aga gc | 5′ acg tgc atg gcc act agg |
| | (CX26CD1S) | (CX26-482R) |
| 651 bases | 5′ gca ttc gtc ttt tcc aga gc | 5′ cag gat gca aat tcc aga ca |
| | (CX26CD1S) | (CX26-810R) |
| 864 bases | 5′ gca ttc gtc ttt tcc aga gc | 5′ ggc cta cag ggg tttc aaa t |
| | (CX26CD1S) | (CX26CD3 |

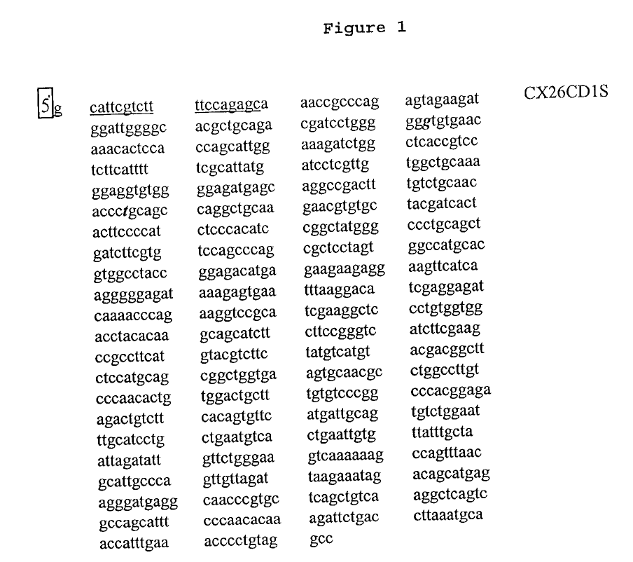

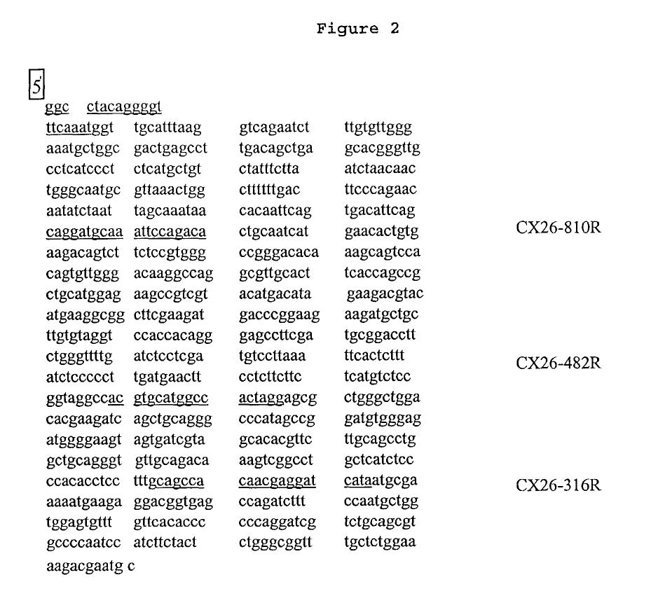

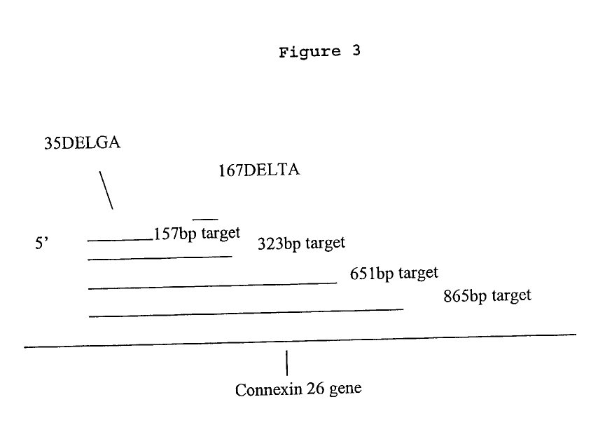

The PCR products generated were 157, 323, 651 and 864 bases in length and they were perfectly complementary to probes 35DELGS, 35DELGA, 167DELTS and 167DELTA. For example, the 864 base PCR product sense strand hybridized with both 35DELGA and 167DELTA, while the antisense strand hybridized with 35DELGS and 167DELTS. The 157 base oligonucleotide molecule (sense and antisense strands) only hybridized with 35DELGS and 35DELGA because this oligonucleotide was too short to cover both mutation regions. FIGS. 1 and 2 show the sequence of sense and antisense strands of the 864 base long PCR products respectively. FIG. 3 shows the location where the complementary probes hybridized to these targets.

Cy5 labeled PCR products were generated using Cy5 end-labeled PCR primers for detection after hybridization. In addition, Cy5 molecules were conjugated to the target molecules to optimize the signal. This was accomplished by preparing biotinylated PCR products followed by the addition of streptavidin-Cy5. All four biotinylated PCR products were generated by adding some quantity of biotinylated dCTP during the PCR reaction. A 20:80 (biotin:non-biotin) ratio was used since it produced the highest amount of PCR products. PCR was performed using a PerkinElmer 9600 PCR device (PerkinElmer, Norwalk, Conn.), and the PCR products were purified and concentrated using Qiagen kits (Valencia, Calif.).

B. Preparation of Microarray Supports

Previous experiences showed that APTES (3-aminopropyltriethoxysilane) and PDC (1,4-phenylene diisothiocyanate)-coated glass slides were appropriate for attachment of probes for heterogeneous hybridization.

Preparation of Slides for Passive Hybridization:

In a clean hood, a Teflon wafer carrier (Fluoroware, Chaska, Minn.) was loaded with twenty-four 25 mm×75 mm glass slides. A 750 ml solution of 30% (w/w) hydrogen peroxide and 96% (w/w) sulfuric acid in a 1-2 ratio by volume was prepared by adding acid to the hydrogen peroxide in a glass beaker. The solution was heated to 120° C. on a hot plate before the wafer carrier was immersed in the solution. The temperature was maintained at 120° C. for 10 minutes. The carrier was transferred to another beaker containing deionized water and rinsed for 5 minutes. The rinsing process was then repeated three times with clean water each time. The slides were dried in a clean oven at 110° C. for 5 minutes.

Preparation of Glass Slides for Electrical Hybridization:

Indium/Tin-Oxide-coated (ITO) glass slides were prepared commercially (Delta Technologies, Stillwater, Minn.). The ITO-coated slides were carefully cleaned by the manufacturer and, when maintained in a clean environment, were used “as is”.

Silanization (APTES Coating):

150 ml of solution containing 1% (v/v) APTES (3-aminopropyltriethoxysilane) (Sigma, St. Louis, Mo.) in 95% (v/v) ethanol in water was prepared for silanizing the glass slides. After mixing the solutions, the silanization solution was titrated to pH 7.0 by adding acetic acid. A slide holding rack capable of holding twenty slides, either pre-cleaned or ITO-coated, was immersed in the solution in a staining beaker for twenty minutes at room temperature. Parafilm was used to seal the container to prevent the solution from absorbing moisture. After silanization, the slides were rinsed in fresh 100% ethanol at room temperature three times and then cured in a clean oven at 110° C. for twenty minutes or cured at room temperature for twenty-four hours.

1,4-Phenylene Di-isothiocynate Modification:

Silanized slides were treated with 0.2% (w/v) PDC (1,4-phenylene diisothiocyanate) (Sigma, St. Louis, Mo.) in 10% (v/v) pyridine/90% dimethylformamide (Fisher) at room temperature for two hours. The staining beaker was sealed with Parafilm to prevent the solution from absorbing moisture. The slides were washed with HPLC-grade methanol and acetone, each for five minutes at room temperature and then the slides were dried in a clean oven at 110° C. for five minutes.

C. Spotting

In order to generate DNA chips, a custom arrayer was built. This moderate-cost, easy-to-build arrayer was capable of holding thirty-two 1″×3″ slides. It was also designed to hold two 96 or 384-well microtiter plates. Using this arrayer, the deposition tip was positioned with 25 μm precision. This one-tip deposition arrayer generated 32 identical slides, each containing up to 96 different sample spots, and was capable of depositing spots 500 μm apart in volumes of 5 nl.

Oligonucleotide probes at concentrations of 100 μM were mixed 1:1 with Micro-Spotting solution (TeleChem International Inc, Sunnyvale, Calif.). Probes were spotted robotically by the arrayer at a volume of 5 nl and at a spacing of 500 μm from center to center. Each probe was spotted in duplicate spots in the same row in order to check the uniformity of deposition. The spotted slides were left at room temperature overnight in Petri dishes with moisture present to aid the chemical linkage of the probes to the surface.

D. Washing and Blocking

After incubating overnight for chemical linkage between the probes and glass surface, the microarray was washed to remove the unlinked probes. Spotted slides were first washed individually with 10 ml of pH 8 1× TE buffer and then washed with 10 ml of deionized water three times. The slides were then put in a 20-slide-holding-rack and washed in 55° C. deionized water for 15 minutes. After the slides were dried in a clean hood, the rack was immersed in a staining beaker with 150 ml 1 M Tris-HCL (pH 7.5) for 1 hour. The slides were then washed individually with 10 ml of 10 M NaCl followed by 10 ml of deionized water. These steps were performed at room temperature.

E. Hybridization Reactions

25 μl of hybridization solution containing a known quantity of target molecules were hybridized to the probe arrays. When PCR products were used, products were denatured into single stranded PCR target molecules before hybridizing with the probes. This was achieved by heating the hybridization solution to 95° C. for ten minutes and then “snap chilling” on ice for five minutes.

Electric-Field Hybridization:

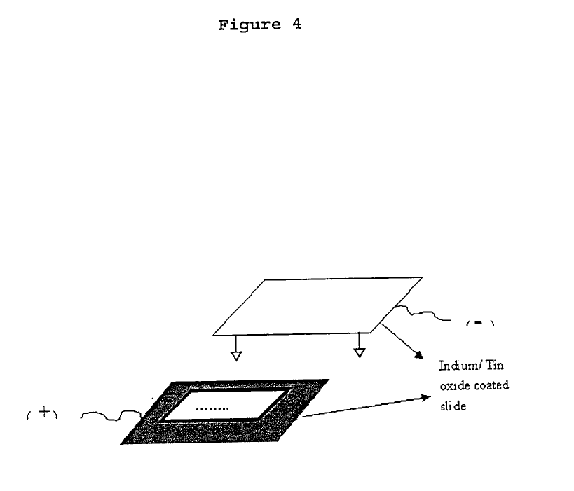

A double-sided adhesive barrier (MJ Research, Watertown, Mass.) with thickness 300 μm and a square area of 81 mm² was attached to the ITO-coated glass surface to surround the probe area thereby defining a reaction space. Hybridization solution (50 mM Histidine, 1 M NaCl, and 1 mM CTAB) containing target molecules was then pipetted into the chamber and a second ITO-coated slide was put in place to enclose the reaction space. The ITO-coated surfaces faced each other and acted as positive and negative electrodes for creating the electric-field which effected transport of the target molecules electronically through the solution (FIG. 4). The slides were left on the heating block maintained at the desired temperature. The electric-field was applied in one of two ways: either during the hybridization to improve the speed of hybridization or after passive hybridization to enhance discrimination efficiency. After applying the electric-field, the slides were separated, the barrier was removed and the slides were washed with deionized water. The array was then labelled with 25 μl of 6× SSPE and 1 mM CTAB staining solution containing 250 ng streptavidin Cy5 and 2.5 ng acetylated BSA. The staining solution was pipetted onto the array and covered with a 25 mm×25 mm cover slip and left on the heating block maintained at the same temperature as used for hybridization for 30 minutes before the cover slip was removed. The array was then washed with a solution of 6× SSPE and 1% SDS at the hybridization temperature for 15 minutes. The slide was then rinsed in deionized water twice before drying in a clean hood at room temperature.

Passive Hybridization:

Target molecules were mixed with hybridization solution consisting of 6× SSPE and 1 mM CTAB. This solution was then placed in contact with the probe array in a sealed chamber (FIG. 4). A double-sided adhesive barrier with a thickness of 300 μm and a square area of 81 mm² was again attached to the glass surface to surround the probe area. Hybridization solution containing target molecules was then pipetted into the reaction space and sealed with a plastic cover (provided in the same package as the barrier) to prevent evaporation. The slides were left on a heating block maintained at the desired temperature, but without application of an electrical potential across the spaced apart electric films. After incubation, the chamber was removed and the array was rinsed in deionized water at room temperature. The slides were then stained with Cy5, as described previously for Electric-Field Hybridization.

F. Scanning and Data Acquisition

Fluorescently labeled arrays were scanned to quantitate the degree of hybridization. The slides were scanned using a ScanArray 5000 device (GSI Lumonics, Watertown, Mass.).

II. Results:

According to the speed of hybridization results obtained from the passive hybridization reactions described above, hybridization reactions using a target quantity of 100 fmole in 25 μl of hybridization solution performed within a hybridization chamber (thickness=300 μm, area=81 mm²) at 48° C. will show detectable hybridization results for a 157 base target within 4 hours. Similarly, 10 hours is required for a 323 base target, 14 hours for a 651 base target, and 24 hours for a 864 base target. To obtain a much higher, and more easily distinguished hybridization signal will require an even longer period of time.

A. Electric-Field Hybridization Speed

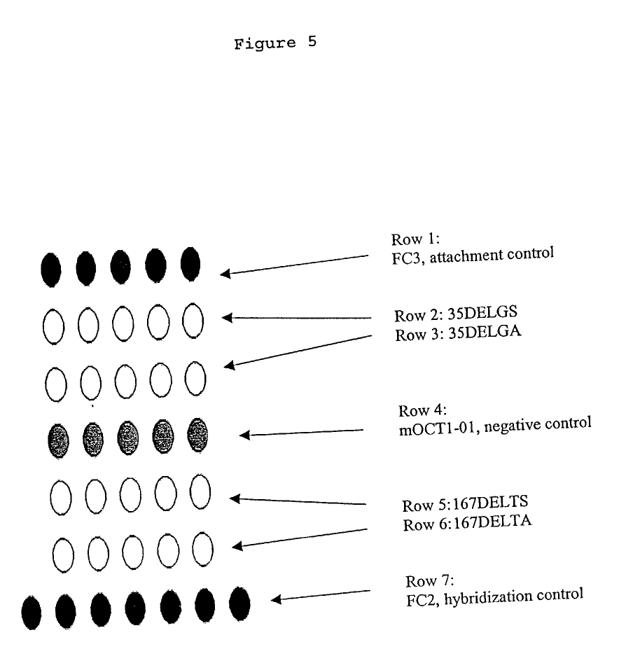

In order to overcome the slow hybridization process of passive hybridization, an electric-field was established to accelerate the hybridization reaction. Since DNA is negatively charged, applying an electric-field in the desired direction of target diffusion facilitated the transport of the target molecules, just as in DNA electrophoresis. Electric-field hybridization reactions were carried out using four probes, 35DELGS, 35DELGA, 167DELTS, 167DELTA, to capture their complementary Connexin 26 PCR targets of various lengths (157, 323, 651, and 864 bases). The probe oligonucleotides (20 nt) were spotted on and chemically linked to ITO-coated glass slides. FIG. 5 shows the spotted DNA microarray pattern. Each probe was spotted in duplicate in the same row in order to check the uniformity of deposition. The hybridization control probe, FC2, was spotted in the last row (Row 7), and was spotted as seven spots (instead of 5 spots as in the other rows) to create a non-symmetric pattern for orientation. The attachment control probe, FC3, was spotted in Row 1 and the negative control, mOCTl-01, was spotted in Row 4. Probes 35DELGS and 35DELGA were spotted in Rows 2 and 3, respectively. Rows 5 and 6 were spotted with probes 167DETS and 167DELTA, respectively.

Biotinylated single-stranded PCR targets (157, 323, 651, and 864 bases) were then hybridized to the probes and stained with streptavidin-Cy5 for signal detection. An electric-field was applied (200 mV) between the slide with the spotted probes and a second conductive slide to bring the targets to the probes rapidly. The target samples were placed between the two slides and the field was applied for between 1.5 and 60 minutes. All hybridizations were performed at 48° C. This temperature was at least 5 degrees lower than the melting temperature of any hybrids that should form on the surface, so all complementary targets theoretically had an equal likelihood of forming duplexes with their probes at the surface. The hybridization apparatus was then disassembled and the hybridization results quantitated by detecting labeled hybrids using a laser scanner. The results compared to the passive hybridization reaction times are provided in Table III:

TABLE III

| Size of PCR | Hybridization Time | Hybridization Time |
|---|---|---|
| Product (nt) | (no Electric-Field) | (with Electric-Field) |
| 157 | 7.9 hours | 10.5 minutes |
| 323 | 11.1 hours | 10.5 minutes |
| 651 | 16.4 hours | 11.5 minutes |
| 864 | 32.9 hours | 60 minutes |

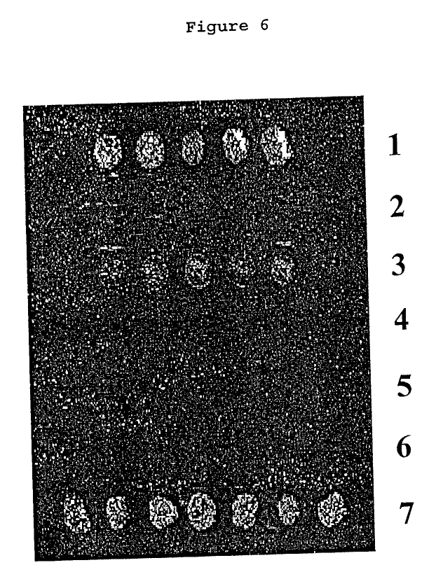

The scanning results are also illustrated in FIGS. 6 through 9. FIG. 6 shows the result of the 157 base target electric-field hybridization reaction after 9 minutes, a case that required at least 4 hours using passive hybridization. In this very short time period, compared to hours in passive hybridization, a hybridization signal was obtained from the 35DELGA probe (FIG. 6, Row 3) and both positive controls, but no negative control signal.

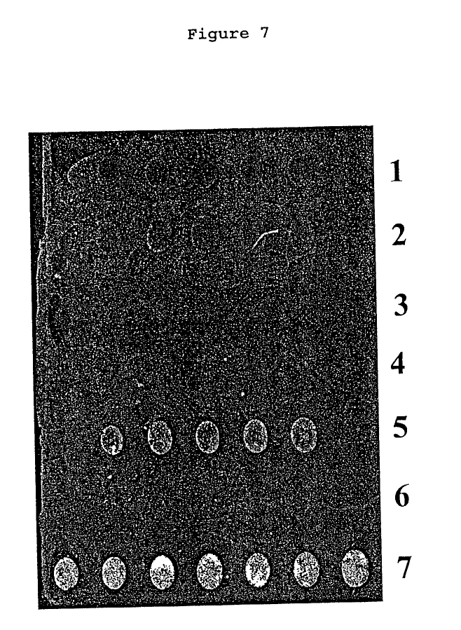

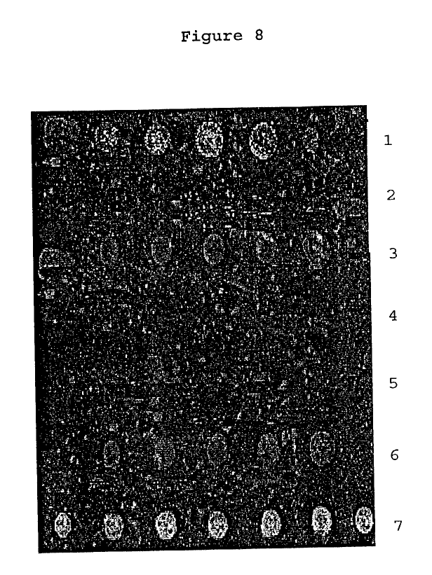

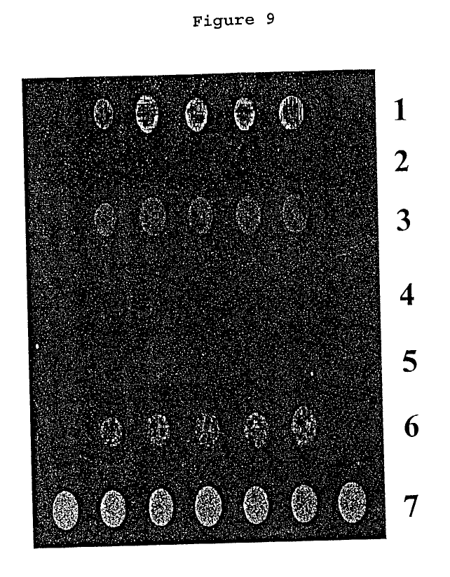

FIG. 7 shows the result of the 323 base target electric-field hybridization reaction after 9 minutes (which required 10 hours with passive hybridization); FIG. 8 shows the result of the 651 base target after 10 minutes (which required 14 hours for a weak signal with passive hybridization); and FIG. 9 provides the result of the 864 base target after 60 minutes (which required more than 24 hours for a signal with passive hybridization).

The results demonstrate that electric-field enhanced hybridization is much faster (approximately 10 to 60 minutes) than passive hybridization (8 to 24 hours). One potential explanation for these results is that the electrical field sets up a concentration gradient that decays exponentially from the surface to bulk solution such that the diffusion away from the surface balances the movement toward the surface. This theory predicts enhancements from about 40 to 200 times for DNA in the size ranges tested above.

The only significant difference between predicted and actual improvements in time are for the largest size fragment (864 base). The time for this fragment should be considerably less than 60 minutes shown in the last column of the last row in Table III.

B. Passive Hybridization Speed

In order to determine the relationship between target length and hybridization time, the probes used in the Electric-Field Hybridization reactions were spotted onto chemically treated glass slides using volumes of 5 nl and a spacing of 500 μm from center to center of each spot.

After the slides were washed, a 25 μl hybridization solution containing 100 fmole of FC3A (hybridization control target) and a certain quantity of one of the four sizes of PCR products was sealed with one of the slide microarrays to initiate passive hybridization. After incubating the array to facilitate hybridization, the array slides were washed, stained with streptavidin Cy5 and scanned.

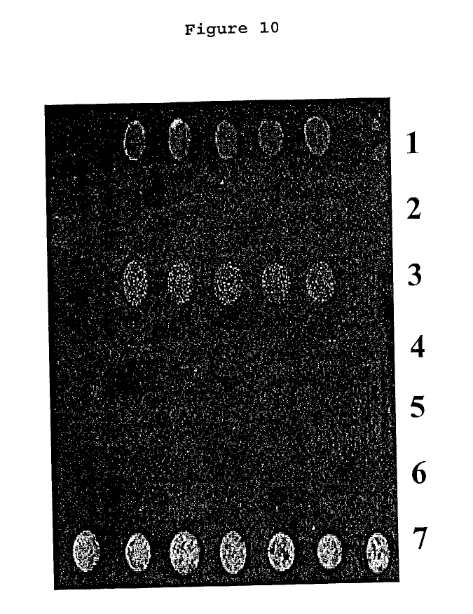

FIG. 10 shows the scan of the 157 base target hybridization reaction after 4 hours. Both the positive and negative controls reacted as expected. The 157 base PCR product contained 2 different strands, sense and antisense, which were supposed to hybridize with probes 35DELGA and 35DELGS, respectively. However, hybridization was only detected between the sense strand and 35DELGA (Row 3).

### EXAMPLE 2

### Discrimination Between Perfectly Matched and Single-Base Mismatched Hybrids Using Reversed Electric-Field Hybridization

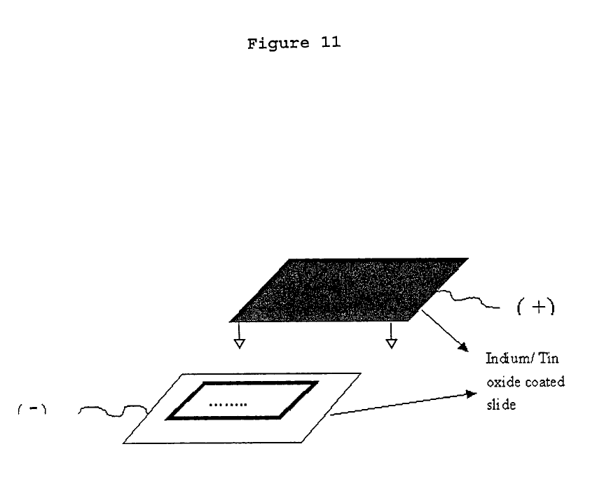

Applying an electric-field to the hybridization reaction was further used to improve the discrimination between perfectly matched hybrids and those containing a single-base mismatches. This method involved applying the electric-field in the reverse direction for a short time (a few seconds) after application in the forward direction. Imperfect matches that did not bind as tightly as the perfect matches were removed more easily from the positive electrode slide by the reverse electric-field. Since hybrids on the surface are negatively charged, the targets were repelled from the surface, breaking the weaker hydrogen bonds between the probes and the target molecules, but leaving the stronger covalent bonds intact between the probes and the glass surface. FIG. 11 illustrates the reverse electric-field hybridization device.

To demonstrate the effects of reversed electric-field, passive hybridization was performed for 5 minutes followed by the application of the reversed electric-field in the following manner: an electric-field strength of 6.67V/cm was applied to the probe slide, 1 minute negatively charged, then 1 minute positively charged, repeating the process 5 times, then 15 seconds negatively charged. The one minute negative charge was to denature the hybrids and then the target was brought back to the surface to react with the probe again. After repeating this process several times, the percentage of perfectly matched hybrids was greater than the single-base mismatched hybrids.

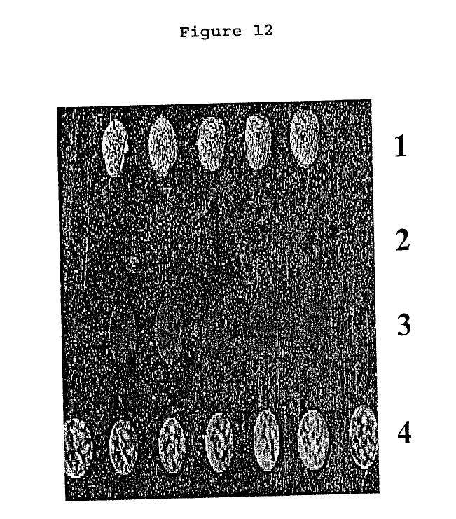

FIG. 12 shows the results of a reversed electric-field hybridization reaction where passive hybridization was performed using ITO-coated slides at 52° C. for 5 minutes, followed by reversed electric-field treatment. Row 2 was the negative control (no hybridization detected), and row 4 was the attachment control. Signal from Row 1 (perfectly matched hybrids) was approximately 3.1 fold higher compared to Row 3 (single-base mismatched hybrids).

By applying a reversed electric-field, the matched to mismatched intensity ratios were enhanced by a factor of 2 to 3 over the non-field case, a significant improvement over hybridization alone. Such improvements may be used to advantage to generate more accurate hybridization results. In addition, such processes may be adapted in diagnostic kits to facilitate the rapid and accurate detection of genetically-linked hematologic disease states.

While certain of the preferred embodiments of the present invention have been described and specifically exemplified above, it is not intended that the invention be limited to such embodiments. For example, the solid support and barrier may be molded as an integral unit. Various modifications may be made thereto without departing from the scope and spirit of the present invention, as set forth in the following claims.

What is claimed is:

1. A device for rapidly performing nucleic acid hybridization reactions, said device comprising a solid support and a continuous barrier disposed on and surrounding a predetermined surface area of said solid support, the barrier and the predetermined surface area of the solid support surrounded by said barrier defining a reaction space within said barrier, at least a portion of the support surface within said reaction space bearing a first electrode comprising a coating of electrically conductive material and a micro-array of nucleic acid probes; a removable cover having a surface and cooperating with said barrier to enclose said reaction chamber, at least a portion of the surface of said cover within said reaction space bearing a second electrode comprising a coating of electrically conductive material; and a source of electric potential including a positive pole and a negative pole, with said positive pole connected to said first electrode and said negative pole connected to said second electrode.

2. The device as claimed in claim 1, wherein said solid support and said cover are transparent.

3. The device as claimed in claim 2, wherein each said coating of electrically conductive material is transparent.

4. The device as claimed in claim 3, wherein each said coating of electrically conductive material comprises indium/tin oxide.

5. A method for rapidly performing nucleic acid hybridization reactions comprising the step of:
a. providing a device as claimed in claim 1;
b. depositing into the reaction space of said device a volume of test sample suspected of containing target nucleic acid molecules complementary to said nucleic acid probes;
c. enclosing said reaction space with said cover; and
d. applying an electrical potential across the electrodes of said device, the first electrode being positive and the second electrode being negative.

6. The method as claimed in claim 5, further including detecting the occurrence of hybridization reactions between said nucleic acid probes and said target nucleic acid molecules.

7. A method for discriminating between hybrids formed by the reaction between (i) a nucleic acid probe and a target nucleic acid molecule that is perfectly matched to said nucleic acid probe and (ii) said nucleic acid probe and a target nucleic acid that differs from said nucleic acid probe by at least one mismatched base pair, comprising the steps of:
a. providing a device as claimed in claim 1;
b. depositing into the reaction space a volume of test sample containing target nucleic acid molecules, said target nucleic acid molecules comprising said perfectly matched nucleic acid and said nucleic acid having at least one mismatched base pair;
c. subjecting the contents of said reaction space to conditions promoting hybridization between said nucleic acid probes and said target nucleic acid molecules;
d. enclosing said reaction space with said cover;
e. applying a potential difference to said electrodes, the first electrode being positive and the second electrode being negative, said potential difference being applied for a time sufficient to effect disassociation of a fraction of the hybrids formed in step c;
f. reversing the potential difference applied to said first and second electrodes in step e;
g. restoring the potential difference established in step e; and
h. determining the level of hybrids formed between said nucleic acid probe and said perfectly matched nucleic acid in relation to hybrids formed between said nucleic acid probe and said nucleic acid having at least one mismatched base pair and comparing said level to the corresponding level resulting from step c.
